(19) United States
(12) Patent Application Publication
(10) Pub. No.: US 2019/0175610 A1
(43) Pub. Date: Jun. 13, 2019

(54) FORMULATION OF GALANTAMINE AND CARNITINE AND METHOD OF FATTY ACID MOBILIZATION

(71) Applicant: GLYKON TECHNOLOGIES GROUP, LLC, Seattle, WA (US)

(72) Inventors: Dallas L. CLOUATRE, Seattle, WA (US); Daniel E. CLOUATRE, Seattle, WA (US)

(73) Assignee: GLYKON TECHNOLOGIES GROUP, LLC, Seattle, WA (US)

(21) Appl. No.: 15/905,512

(22) Filed: Feb. 26, 2018

Related U.S. Application Data
(60) Provisional application No. 62/595,728, filed on Dec. 7, 2017.

(51) Int. Cl. A61K 31/55; A61K 31/205; A61P 3/04; A23L 33/105; A23L 33/175

(52) U.S. Cl. CPC A61K 31/55; A61K 31/205; A23L 33/15; A23L 33/105; A23L 33/175; A61P 3/04

(57) ABSTRACT

A method of reducing abdominal and subcutaneous fat in the subject as disclosed whereby the subject is administered a formulation comprised of a combination of galantamine and L-carnitine on a daily basis of a period of days.

### FIELD OF THE INVENTION

The present invention relates to a formulation and its use in a method for enhancing metabolism in general and specifically fat mobilization. The formulation is useful in metabolic fitness, to reduce abdominal and subcutaneous fat and is administered as a dietary supplement containing an herbal extract or other preparation containing galantamine and a source of L-carnitine.

### BACKGROUND OF THE INVENTION

Galantamine is an alkaloid that is obtained from the bulbs and flowers of Galanthus nivalis (also known as Galanthus caucasicus, Caucasian or Voronov's snowdrop), Leucojum aestivum (snowflake), Lycoris radiata (Red Spider Lily), Galanthus woronowii (Amaryllidaceae), Narcissus (Daffodil) and related species. It also can be prepared synthetically. Regardless of the source, in practice the usual active ingredient is the salt galantamine hydrobromide.

Galantamine belongs to a class of compounds known as cholinesterase inhibitors. These compounds, both natural and synthetic, inhibit the activity of the enzyme acetylcholinesterase. Galantamine is a competitive and reversible inhibitor. As an acetylcholinesterase inhibitor, galantamine works by enhancing cholinergic function though increasing the local concentration of neurotransmitter acetylcholine in the brain and other tissues. Galantamine further exhibits activity in modulating the nicotinic cholinergic receptors, including in the nerves activating muscle tissues, to either increase acetylcholine release or improve nerve reactivity. Galantamine activates the muscle-type acetylcholine receptor by interacting with a binding site that is distinct from the site for nicotinic agonists.

A drug based upon this naturally-occurring compound is used for the treatment of mild to moderate Alzheimer's disease and some other forms of dementia, a use that has been known perhaps since the time of the Greek poet Homer. U.S. Pat. No. 6,159,476 teaches that galantamine, as part of an elaborate formula containing nine other ingredients, may improve the “effectiveness of muscle performance in an athlete” with reference to evidence that “administration of galanthamine for 3 weeks increases the strength and the endurance of active athletes.” U.S. Pat. No. 8,603,546 similarly teaches that galantamine itself under a very specialized dosage and administration regimen leads to an improvement that “comprises greater muscle strength, endurance, speed, or a combination thereof.”

Galantamine was sold in the United States from 1980-1985 as a component of Energix, “an imported biostimulant” sold for “increased loading of the muscular and nervous systems; in physical and mental fatigue; for recovery . . . after major physical loading.” (Country Life Energix Brochure; also “The Plant Alkaloid Galantamine-Approved as a Drug; Sold as a Supplement” [2001]).

Galantamine is not known for improving fatty acid metabolism or weight loss. In relatively healthy subjects with metabolic syndrome taking part in a randomized double-blind, placebo-controlled trial, oral galantamine at 8 mg daily for 4 weeks followed by 16 mg daily for 8 weeks versus placebo did not lead to significant changes in body composition. (Consolim-Colombo F M, Sangaleti C T, Costa F O, et al. Galantamine alleviates inflammation and insulin resistance in patients with metabolic syndrome in a randomized trial. JCI Insight. 2017 Jul. 20; 2(14). pii: 93340.) Extrapolations based on weight improvements with obese mice had suggested that galantamine might improve body weight and visceral adiposity. (Satapathy S K, Ochani M, Dancho M, Hudson L K, et al. Galantamine alleviates inflammation and other obesity-associated complications in high-fat diet-fed mice. Mol Med. 2011; 17(7-8):599-606.) Actual clinical work performed in human subjects most likely to benefit demonstrated that galantamine by itself does not provide these benefits even at significant dosages taken for an extended period of time.

L-carnitine is an amino acid supplied in the diet primarily through animal muscle meats (from those of sheep and lamb, in particular), and also is manufactured in the body, mainly in the liver and the kidneys. Produced from the essential amino acid lysine, the body's synthesis of L-carnitine requires the vitamins C, B-6 and niacin, along with iron and the amino acid methionine. In humans L-carnitine is concentrated in the heart and the skeletal muscles, and also in the brain and in the sperm. The primary role of L-carnitine in the body is as a biocatalyst. It serves to transport fatty acids across the membrane of the cell and into the mitochondria, where these fatty acids are metabolized for energy. It also aids in the removal of waste products from the mitochondria. Benefits have been demonstrated for sperm quality, symptoms of intermittent claudication, blood glucose levels and a number of other areas, albeit generally considered to be minor.

At two or more grams per day, L-carnitine in humans improves recovery capacity, mitigates metabolic stress and muscle soreness of exercise, increases androgenic response to exercise and reduces exercise-induced muscle tissue damage. There is little evidence that L-carnitine supplementation improves athletic performance otherwise in healthy individuals either anaerobic or aerobic performance. (Brass E P. Carnitine and sports medicine: use or abuse? Ann N Y Acad Sci. 2004 November; 1033:67-78.)

Rodent trials have been published showing effects on fat loss. However, despite decades of studies, there is little or no evidence of significant effects of L-carnitine in humans except, possibly, in the elderly. (Saper R B, Eisenberg D M, Phillips R S. Common dietary supplements for weight loss. Am Fam Physician. 2004 Nov. 1; 70(9):1731-8.)

Direct tests with athletes looking specifically at fatty acid metabolism have found no benefit. (Broad E M, Maughan R J, Galloway S D. Carbohydrate, protein, and fat metabolism during exercise after oral carnitine supplementation in humans. Int J Sport Nutr Exerc Metab. 2008 December; 18(6):567-84.) This lack of benefit is in line with the known fact that only increases in muscle carnitine content on the order of 25-50 percent, something extremely difficult to accomplish, will influence muscle fuel choice and metabolism.

A clinical trial combining L-carnitine with choline yielded ambiguous results with regard to effects on fat metabolism and no changes in body composition. Nineteen women were placed in three groups: 1) placebo, choline or L-carnitine preloading for period of 1 week followed by supplementation with choline plus L-carnitine during week 2 through week 3. All groups exercised in week 3. Body fat percentage did not change from baseline after treatments on day 21 nor at follow-up on day 35. Although a blood marker for fatty acid mobilization increased, results indicated incomplete oxidation of fatty acids and disposal of their carbons in urine as acylcarnitines, i.e., no indication of a true improvement in fat metabolism nor of greater energy generation from fatty acids. (Hongu N, Sachan D S. Carnitine and choline supplementation with exercise alter carnitine profiles, biochemical markers of fat metabolism and serum leptin concentration in healthy women. J Nutr. 2003 January; 133(1):84-9.)

In general publications emphasize the effect of galantamine on cholinesterase activity, nerve function, the lowering of the excitation threshold and the speed of muscle contraction as well as improvements in insulin resistance and certain markers of inflammation. Likewise, L-carnitine has demonstrated benefits for intermittent claudication, blood glucose levels and some aspects of sports recovery. Neither galantamine nor carnitine in humans, as opposed to animal trials has demonstrated significant benefits in the area of visceral or subcutaneous fat reduction nor in fatty acid metabolism beyond the known physiologic role of carnitine in the transfer of fatty acids into the mitochondria and related function. Likewise, the combination of carnitine plus choline has not been shown to reduce body fat percentage or body composition, nor an improvement in fatty acid metabolism per se as indicated, for example, in improved exercise performance.

### SUMMARY OF THE INVENTION

An aspect of the invention is an oral formulation comprising a therapeutically effective amount of two components which are galantamine and L-carnitine. The formulation may be a liquid formulation which includes flavoring and coloring agents and an excipient or carrier which may be water. The formulation may be in the form of pills, tablets, capsules, or a dry powder which can be added to a liquid just prior to administration to create a liquid formulation.

The method of the invention includes use of the formulation in the treatment of subjects which have been diagnosed with excess abdominal and/or subcutaneous fat. The method comprises the administration of the formulation of the invention such that from 500 mg to 6 g of the L-carnitine are administered per day and 2 mg to 32 mg of the galantamine are administered per day.

The method of the invention includes use of the formulation in the treatment of subjects which have been diagnosed with excess abdominal and/or subcutaneous fat. The method comprises the administration of the formulation of the invention such that from 1 g to 5 g of the L-carnitine are administered per day and 4 mg to 20 mg of the galantamine are administered per day.

The method of the invention includes use of the formulation in the treatment of subjects which have been diagnosed with excess abdominal and/or subcutaneous fat. The method comprises the administration of the formulation of the invention such that from 2 g to 4 g of the L-carnitine are administered per day and 8 mg to 16 mg of the galantamine are administered per day.

The method of the invention includes use of the formulation in the treatment of subjects which have been diagnosed with excess abdominal and/or subcutaneous fat. The method comprises the administration of the formulation of the invention such that from 2 g of the L-carnitine are administered per day and 4 mg of the galantamine are administered per day.

The formulation may be administered daily over a period of days, weeks, months or years. The formulation may be administered once a day, twice a day, three times a day, four times a day, etc.

In one aspect of the invention, the formulation is co-administered along with a therapeutically effective amount of an additional compound which may be all or any of vitamin C, vitamin B-6, niacin, iron and methionine. In addition, the composition may be co-administered with ginger in a liquid or solid form.

The present invention shows that galantamine combined with the nutrient L-carnitine enhances fat mobilization and utilization leading to reduced abdominal and subcutaneous fat, a finding with implications not only for athletics, but also for maintaining lean body composition and metabolic fitness. Neither galantamine alone nor L-carnitine alone provides these benefits. Further, adding a choline source to L-carnitine does not provide these benefits, indicating, by extension, that achieving a similar increase in choline via a cholinesterase inhibitor would not be expected to lead to body fat mobilization resulting in reductions in abdominal and subcutaneous fat.

The formulation of the invention which comprises galatamine and L-carnitine enhances fat mobilization and utilization (metabolic fitness) leading to reduced abdominal and subcutaneous fat. The formulation of the invention is administered at a dosage range based on the lower bounds of adequate intake (determined by pharmacokinetics) of galantamine and L-carnitine and the upper bound of galantamine before side effects become significant.

Galantamine is active in a range of 2 mg to 16 mg per day, with therapeutic doses for dementia often in the range of 24 to 32 mg. Intakes above 16 mg per day may provide greater benefits. However, these benefits may be outweighed by the rapidly escalating rate of gastrointestinal and other side effects when higher amounts are administered. Side effects are similar to placebo until the initial dosage reaches 8 mg per day, at which point significantly more individuals are affected. Although the percentage still is not large, sensitive individuals may experience minor side effects, such as headache and diarrhea, escalating with dosage above this point. Tolerance to galantamine side effects develops with continued usage. L-carnitine is effective at intakes starting near 500 mg per day and ranging to more than 4 grams per day, with a diminishing marginal rate of improvement above the 4 gram level of intake.

An aspect of the invention is the use of a composition for achieving a reduction in abdominal and subcutaneous fat wherein the composition is comprised of a combination of galantamine and L-carnitine.

Another aspect of the invention is a use as described here wherein the wherein the galantamine is derived from either natural or synthetic sources, such as Galanthus nivalis, Leucojum aestivum, Lycoris radiata, Galanthus woronowii, Narcissus and the salt galantamine hydrobromide.

Another aspect of the invention is a use as described here wherein the L-carnitine is supplied in the form of L-Carnitine, Acetyl L-Carnitine, L-Carnitine Fumarate, L-Carnitine Tartrate, Glycine Propionyl L-Carnitine or other L-carnitine formats.

Another aspect of the invention is a use as described here wherein the the galantamine is ingested in amounts averaging from 2 mg to 32 mg per day.

Another aspect of the invention is a use as described here wherein the L-carnitine is ingested in amounts averaging from 500 mg to 6 grams per day.

Another aspect of the invention is a use as described here wherein the the combination is consumed one to two hours prior to exercise.

These and other aspects, objects, advantages, and features of the invention will become apparent to those persons skilled in the art upon reading the details of the formulation and method as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a graph showing how body mass index (BMI) is calculated with respect to individuals based on their height and weight, showing the calculations with respect to both pounds and kilograms and meters and feet.

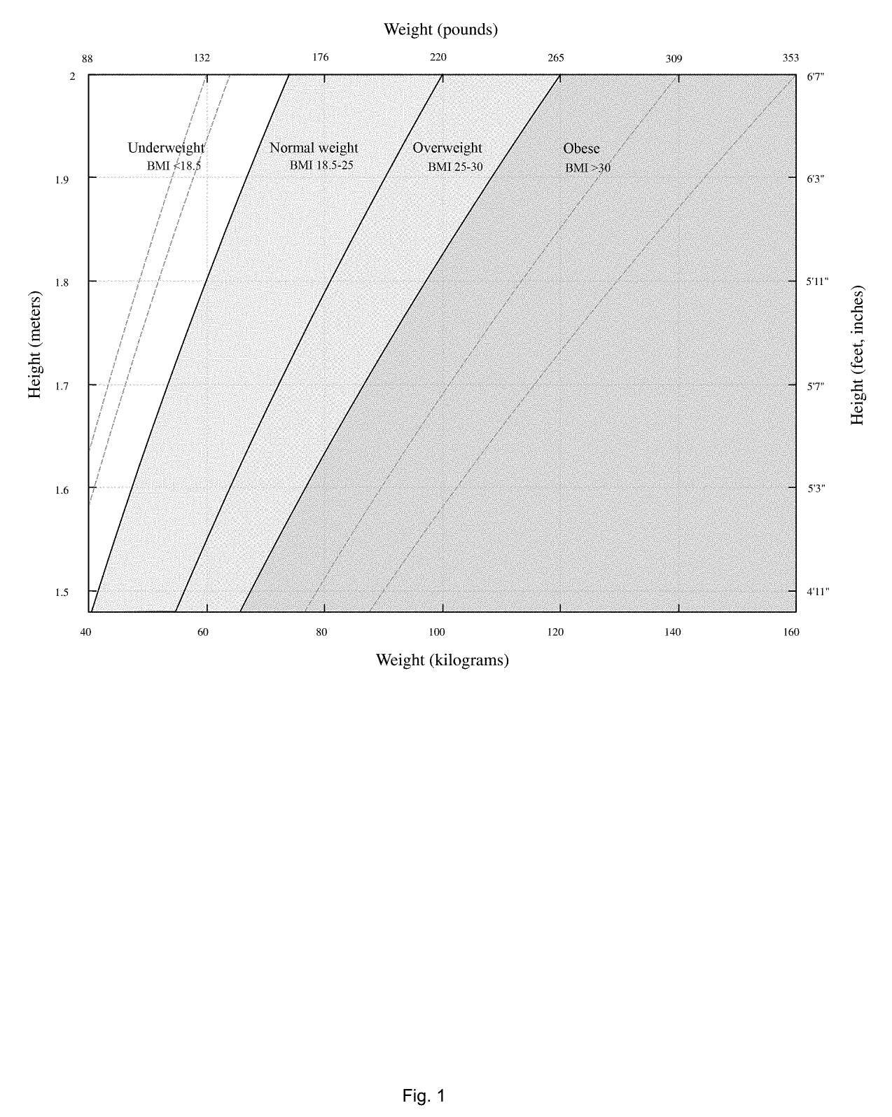

### DESCRIPTION OF THE PREFERRED EMBODIMENTS

Before the present formulation and methods are described, it is to be understood that this invention is not limited to particular components and steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a excipient” includes a plurality of such excipients and reference to “the dose” includes reference to one or more doses and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Invention in General

The invention includes a method of treatment wherein the patient is first diagnosed as having a body mass index (BMI) in excess of 25 based on units of kg/m². The BMI may be at 26, 27, 28, 29, 30 or more. After diagnosing the subject, the formulation of the invention is administered to the subject at a therapeutically effective amount, wherein the formulation is comprised of a combination of galantamine and L-carnitine. The formulation is repeatedly administered as needed over a period of days, weeks, months or years, thereby achieving a reduction in the abdominal and subcutaneous fat of the subject and reducing the subjects BMI by 0.5 kg/m² or more, 1 kg/m² or more to 2 kg/m² or more to 3 kg/m² or more to 4 kg/m² or more to 5 kg/m² or more or 6 kg/m² or more.

The formulation may be administered once a day, twice a day, three times a day or more. The formulation may be a liquid formulation wherein the galantamine and L-carnitine are dissolved in the liquid or dispersed in the liquid. The formulation may be a solid powder or may be tablets or capsules. The subject may be dosed in an amount of 2 mg to 32 mg per day of the galantamine and a daily dose from 500 mg to 6 g of L-carnitine.

As used herein, the active ingredient “galantamine” may be in the form of a free base, or its pharmaceutically acceptable salts, solvates (including hydrates), polymorphs, all optical isomers, or combinations comprising at least one of the foregoing forms of galantamine. In addition, the various forms of galantamine can be in crystalline or non-crystalline (amorphous) forms.

As used herein, “pharmaceutically acceptable salts” of galantamine include derivatives of galantamine, wherein galantamine is modified by making non-toxic acid addition salts thereof. Examples of pharmaceutically acceptable salts include, but are not limited to, mineral or organic acid addition salts of basic residues such as amines; alkali or organic addition salts of acidic residues such as carboxylic acids; and the like, or a combination comprising at least one of the foregoing salts.

The pharmaceutically acceptable salts include salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like. Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, and the like. Combinations comprising at least one of the foregoing salts may also be used.

A particularly useful salt of galantamine is galantamine hydrobromide (1:1).

L-carnitine, also known as. γ-trimethylamino-β-hydroxybutyrate or Vitamin B_{T}, is a normal endogenous intermediary metabolite present in blood, urine and all cells of animals. L-carnitine has dual functions in lipid metabolism, transporting fatty acids and other acylated compounds across the inner mitochondrial membrane and maintaining the acyl CoA/free CoA ratio between the mitochondria and the cytosol. Carnitine readily forms esters with CoA compounds and thus can remove such compounds from the cell during abnormal metabolic conditions.

Deficiency states of carnitine have been described as associated with specific clinical symptoms. These include hepatic dysfunction, encephalopathy, progressive muscle weakness with fatty muscle on biopsy, cardiomyopathy, and failure to thrive.

L-carnitine can be synthesized according to the method described by Tomita et al., J. Physiol. Chem. (1927) 169:263. In addition, it is commercially available from a number of sources including Sigma-Tau (an Italian supplier), Sigma Chemical Co. (St. Louis, Mo.), and Aldrich Chemical Co., Inc. (Milwaukee, Wis.). L-carnitine can be presented as a pure enantiomer or as a mixture of enantiomers (i.e., DL-carnitine).

Forms of L-carnitine useful in conjunction with galantamine include, but are not limited to, L-carnitine, acetyl L-carnitine, acetyl L-carnitine arginate di-HCl, L-carnitine L-tartrate, L-carnitine fumarate and glycine propionyl L-carnitine HCl.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES

### Example 1

Tablets were made with the following formula:

| | |
|---|---|
| L-carnitine fumarate | 2,000 mg |
| Glycine propionyl-L-carnitine HCl | 1,000 mg |
| Galantamine hydrobromide | 7.5 mg |

Seven athletically trained test subjects participated in a 4-week trial. The above formula was taken once a day with a small meal approximately 2 hours prior to working out. Two of the test subjects reported slight tiredness for the first week, but improved strength, stamina and recovery thereafter. In total, 4 out of 7 reported improved strength, 6 out of 7 reported improved stamina, and 7 out of 7 reported better recovery, including not being as sore the next day after extreme workouts. One subject, although reporting a greatly improved rate of recovery from excessive physical training, nevertheless dropped out of the trial due to tightness in the lower back, a development likely related to increased muscle tonus linked to cholinesterase activity. These findings were within the expected parameters. Quite unexpected was the finding that 3 of these already lean athletes reported a noticeable reduction in visible abdominal and subcutaneous fat during the course of the trial. Taken together, these data indicate a significant effect of the combination galantamine and L-carnitine on fat mobilization and body composition.

### Example 2

The following formula adds a choline component as a substrate for other functions of galantamine.

| | |
|---|---|
| Glycine propionyl-L-carnitine HCl | 2,000 mg |
| Choline bitartrate | 1,000 mg |
| Galantamine hydrobromide | 7 mg |

This formula can be delivered via two large tablets or smaller tablets in greater number. Intake can be doubled by those not sensitive to cholinesterase inhibitors. The subject may be treated by the administration of a larger number of smaller capsules. In addition, it is possible to create a dry powder of the formulation, and keep the dry powder in separate individual dosing packages which contain all of the formulation such as the formulation described in the examples below. The dry powder is then added to a liquid such as water just prior to consumption. The dry powder and water or other flavored and or colored liquid may be sold together with the individual dry powder packets.

### Example 3

The following formula adds a choline component as a substrate for other functions of galantamine while utilizing a lower dosage to avoid side effects in individuals who are especially sensitive to galantamine.

| | |
|---|---|
| Glycine propionyl-L-carnitine HCl | 2,000 mg |
| Choline bitartrate | 1,000 mg |
| Galantamine hydrobromide | 4 mg |

This formula can be delivered via two large tablets or smaller tablets in greater number. The dosage can be doubled. As indicated above, different variations of the formulation can be created using different numbers of smaller tablets, dry powder packets including the whole dose which can be added to a liquid, as well as other variations. This is true with respect to each of the formulation examples provided here.

### Example 4

The following formula adds two sources of choline as substrates for other functions of galantamine while utilizing a lower dosage to avoid side effects in individuals especially sensitive to galantamine. Ginger extract is known to be settling to the stomach, hence addresses a primary side effect of cholinesterase inhibitors.

| | |
|---|---|
| Acetyl-L-Carnitine (acetyl-L-carnitine hydrochloride) | 1,500 mg |
| Choline (choline bitartrate) | 750 mg |
| α-GPC (L-alpha glycerylphosphorylcholine) | 250 mg |
| Ginger Extract | 250 mg |
| Galantamine hydrobromide | 4 mg |

This formula can be delivered via two large tablets or smaller tablets in greater number. It is possible with this formulation to take 2 tablets twice per day. After a month of usage, many or even most individuals can take 3 tablets twice per day.

### CONCLUSION

It has been discovered, quite surprisingly in the light of the null results of prior human trials with the individual ingredients, that the combination of galantamine and L-carnitine enhances fat mobilization and utilization (metabolic fitness) leading to reduced abdominal and subcutaneous fat.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of treatment, comprising:
administering to a subject a therapeutically effective amount of a composition comprising a combination of galantamine and L-carnitine; and
thereby achieving a reduction in abdominal and subcutaneous fat in the subject.

2. The method of claim 1 wherein the galantamine is galantamine hydrobromide derived from a sources selected from the group consisting of Galanthus nivalis, Leucojum aestivum, Lycoris radiata, Galanthus woronowii, Narcissus and a salt galantamine hydrobromide.

3. The method of claim 1 wherein L-carnitine is substantially free of D-carnitine, and is in a form selected from the group consisting of L-Carnitine, Acetyl L-Carnitine, L-Carnitine Fumarate, L-Carnitine Tartrate, Glycine Propionyl L-Carnitine and L-carnitine formats.

4. The method of claim 1, further comprising:
repeatedly administering a therapeutically effective amount of a composition to the subject on a daily basis wherein the galantamine is administered in an amount of from 2 mg to 32 mg per day.

5. The method of claim 1 further comprising:
repeatedly administering a therapeutically effective amount of a composition to the subject on a daily wherein the L-carnitine is administered in an amount of from 500 mg to 6 grams per day.

6. The method of claim 1 wherein the composition is administered one to two hours prior to exercise.

7. The method of claim 1, further comprising:
administering a compound selected from the group consisting of vitamin C, vitamin B-6, niacin, iron and methionine.

8. A method of treatment, comprising:
diagnosing a subject as having a body mass index (BMI) of 25 kg/m² or more;
administering to the subject a therapeutically effective amount of a composition comprising a combination of galantamine and L-carnitine; and
thereby achieving a reduction in BMI of the subject of 0.5 1 kg/m² or more.

9. The method of claim 8, further comprising:
repeatedly administering a therapeutically effective amount of a composition to the subject on a daily basis wherein the galantamine is administered in an amount of from 2 mg to 32 mg per day, and wherein the L-carnitine is administered in an amount of from 500 mg to 6 grams per day.

10. The method of claim 8, wherein the galantamine and L-carnitine are combined together in a dry powder in a single dosage package.

11. The method of claim 8, further comprising:
repeatedly administering a therapeutically effective amount of a composition to the subject on a daily basis wherein the galantamine is administered in an amount of from 4 mg to 20 mg per day, and wherein the L-carnitine is administered in an amount of from 1 g to 5 g per day.

12. The method of claim 8, further comprising:
repeatedly administering a therapeutically effective amount of a composition to the subject on a daily basis wherein the galantamine is administered in an amount of from 8 mg to 16 mg per day, and wherein the L-carnitine is administered in an amount of from 2 g to 4 g per day.

13. The method of claim 8, wherein the subject is diagnosed with a BMI of 28 kg/m² or more.

14. The method of claim 8, wherein the subject is diagnosed with a BMI of 30 kg/m² or more.

15. The method of claim 8, wherein the subject is diagnosed with a BMI of 32 kg/m² or more.

16. The method of claim 8, further comprising:
repeatedly administering a therapeutically effective amount of a composition to the subject on a daily basis wherein the galantamine is administered in an amount of from 2 mg to 32 mg per day, and wherein the L-carnitine is administered in an amount of from 500 mg to 6 grams per day until the BMI of the subject decreases to less than 25 Kg/m².
